(19) United States
(12) Patent Application Publication
(10) Pub. No.: US 2004/0203029 A1
(43) Pub. Date: Oct. 14, 2004

(54) Method for the specific fast detection of threadlike bacteria

(76) Inventors: Jiri Snaidr, Munchen (DE); Claudia Beimfohr, Munchen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/735,308

(22) Filed: Dec. 11, 2003

Related U.S. Application Data
(63) Continuation of application No. PCT/EP02/06467, filed on Jun. 12, 2002.

(30) Foreign Application Priority Data
Jun. 12, 2001 (DE) 101 28 400.4

(51) Int. Cl. C12Q 1/68; C07H 21/04

(52) U.S. Cl. 435/6; 536/23.7

(57) ABSTRACT

The invention relates to a method for specific, fast detection of threadlike bacteria, e.g., in activated sludge samples, by in situ hybridization. The invention also relates to oligonucleotide probes which are suitable for use in said method, in addition to kits which enable said detection method to be carried out.

### CROSS REFERENCE TO RELATED APPLICATION

The present application is a continuation of PCT application Ser. No. PCT/EP02/06467, filed Jun. 12, 2002, entitled “Method for Specific, Fast Detection of Threadlike Bacteria,” the disclosure of which is incorporated herein in its entirety; which claims priority from German Patent Application Serial Number 101 28 400.4, filed Jun. 12, 2001, the disclosure of which is incorporated herein by reference in its entirety.

### BACKGROUND OF THE INVENTION

1. Field of the Invention

The present invention pertains to a method for the specific fast detection of threadlike, so-called filamentous bacteria, for example in activated sludge samples, by in situ hybridization. The invention also relates to oligonucleotide probes which are suitable for use in said method, and to kits which enable said detection method to be carried out.

2. Description of the Related Art

Biological wastewater treatment has developed to become a key technology in the area of environmental protection after its introduction at the beginning of this century (1913). In the wake of continuously increasing calls for better water protection and tightening of the legal limits on water discharged from wastewater treatment plants, ever-greater demands are being placed on the effectiveness of this method. Due to the massive algae growth in the North Sea and the Baltic Sea in early summers of 1988 the German legislator is now calling for improved purifying measures to reduce N- and P-containing compounds within the framework of the existing national wastewater regulations of 1 Jan. 1990.

The (presence or absence of the) efficiency of the activated sludge method during wastewater purification is inextricably bound up with the composition of the active bacterial community of the activated sludge in a wastewater treatment plant. To name just one example: filamentous bacteria are a decisive influence on the sedimentation properties of the biomass in the secondary sedimentation tank and thus directly on the functionality of a sewage treatment plant. The presence, and especially the excessive growth, of a variety of filamentous bacteria leads to surface expansion of the activated sludge flocculation, which eventually results in sludge bulking and skimmings. As a result, the purified water can no longer be physically separated from the bacteria of the activated sludge. This leads to bacterial contamination of the drainage ditch and thus of the waters of the neighboring sewage purification plant. Another interruption of operation caused by filamentous bacteria is the formation of excessive foam fractions. These foam fractions are easily carried by the wind, thus also contributing to bacterial contamination of the environment.

Bulking sludge and skimmings as well as the described foam formation are a serious recurring problem in almost half of all sewage treatment plants (Blackbeard et al., A survey of filamentous bulking and foaming in activated sludge plants in South Africa, Water Pollut. Contr. (1986) 85:90-100), which should be prevented or contained by selecting appropriate parameters regarding method and process. A prerequisite for effective control of the purification processes is the acquisition of as much knowledge as possible on the bacterial populations of the activated sludge. In particular, it is crucial to be able to reliably identify the filamentous bacteria which cause these problems by using appropriate techniques.

Our knowledge of the bacteria as the actual protagonists of the wastewater purification process has hitherto been very limited. The term “black box of wastewater purification” is thus frequently heard in this context. The main reason for insufficiency of knowledge in this field is that in the past analysis of bacterial populations of the activated sludge primarily was performed by classical cultivation methods. Despite optimized culture media and cultivation methods, only 1-15% of the bacteria present in the sample may be obtained as monocultures (Wagner et al., Probing activated sludge with proteobacteria-specific oligonulceotides: inadequacy of culture-dependent methods for describing microbial community structure, AppL. Environ. Microbiol. (1993) 59:1520-1525), whereas using this type of detection method up to 99% of the bacteria present in the wastewater remain unidentified. For a long time only simple methods were available for the detection of non-culturable bacteria such as Gram's or Neisser's stains. Due to their morphology and staining behavior, the characterization of bacteria of activated sludge repeatedly leads to wrong interpretations. This is due to the fact that, for example, numerous species among filamentous bacteria have an extremely variable morphology (van Veen et al., Bacteriology of activated sludge, in particular the filamentous bacteria, Antonie van Leeuwenhoek (1973) 39:189-205; Muder and Deinema, 1992, The sheathed bacteria, in: The Procaryotes, Springer Verlag, N.Y.) and also show variable behavior during Gram and Neisser staining (Eikelboom and van Buijsen, 1992, Handbuch für die mikroskopische Schlammuntersuchung, Hirthammer, Müinchen). Error-free identification of these bacteria can thus almost be ruled out.

As a logical consequence of the difficulties arising from traditional methods in the detection of filamentous bacteria, alternative detection methods based on molecular biology therefore would be useful.

In PCR, the polymerase chain reaction, a characteristic segment of the particular bacterial genome is amplified with specific primers. If the primer finds its target site, millions of amplicons of a segment of the genetic information are generated. In the subsequent analysis, using for instance an agarose gel in order to separate DNA fragments, a qualitative evaluation can be made. In the simplest case, this results in the information that the target sites for the primers used were present in the analyzed sample. Other conclusions are not possible, since the target sites may be derived from a living bacterium, a dead bacterium or a naked DNA. Differentiation is not possible here. On the other hand various substances present in the activated sludge may cause inhibition of the DNA amplifying enzyme, the Taq polymerase. This is a frequent cause of false negative results. A further development of the PCR technique is quantitative PCR, in which an attempt is made to create a correlation between the amount of bacteria present and the amount of DNA obtained by amplification. Advantages of the PCR are its high specificity, ease of application and low expenditure of time. Significant disadvantages are its high susceptibility to contamination with consequent false positive results as well as the aforementioned lack of possibility of distinguishing between living and dead cells or naked DNA, respectively, and finally the risk of false negative results due to the presence of inhibitory substances.

However, biochemical parameters are also used for identification of bacteria: The generation of bacteria profiles based on quinone analyzes serves to reflect the bacterial population with as little bias as possible (Hiraishi, A., Respiratory quinone profiles as tools for identifying different bacterial populations in activated sludge, J. Gen. AppL. Microbiol. (1988) 34:39-56). However, this method as well depends on the cultivation of individual bacteria, since the quinone profiles of the monocultured bacteria are required for generating the reference database. In addition, the determination of the bacterial quinone profiles cannot give a real impression of the population distributions actually present in the sample.

In contrast to this, the detection of bacteria by antibodies is a more direct method (Brigrnon, R. L., G. Bitton, S. G. Zam, and B. O'Brien, Development and application of a monoclonal antibody against Thiothrix spp., Appl. Environ. Microbiol. (1995) 61:13-20). Fluorescence-labeled antibodies are mixed with the sample and allow highly specific binding to the bacterial antigens. In the epifluorescence microscope, the bacteria are detected subsequently by their emitted fluorescence. In this way, bacteria can be identified down to strain level. However, three critical disadvantages restrict the application of this method: firstly, monocultures are required for the production of the antibodies. Secondly, the antibody-fluorescent molecule-complex is often large in volume and unwieldy, which generates problems in penetrating the target cells. Thirdly, the detection is often too specific. The antibodies are expensive to produce and frequently detect only one specific bacterial strain, but are unable to detect other strains of the same bacterial species. Frequently, however, strain-specific detection of bacteria is not necessary, but rather detection of a bacterial species or an entire bacterial group is required. Fourthly, production of the antibodies is a relatively tedious and expensive procedure.

### SUMMARY OF THE INVENTION

Some embodiments relate to isolated oligonucleotides. The isolated oligonucleotides can include (i) those described herein, including the oligonucleotides having the sequence of SEQ ID NOs:1-42; (ii) an oligonucleotide being at least 80%, 90%, 92%, 94%, or 96% identical to the oligonucleotides from (i), and may include those rendering possible specific hybridization with nucleic acid sequences of filamentous bacterial cells; (iii) an oligonucleotide differing from one of the oligonucleotides from (i) by a deletion and/or addition, and which may also render possible specific hybridization with nucleic acid sequences of filamentous bacterial cells; and (iv) oligonucleotides hybridizing under stringent conditions with one of the oligonucleotides from (i), (ii) or (iii).

Further embodiments relate to methods for detecting filamentous bacteria in a sample. The methods can include the steps of a) fixing the filamentous bacteria contained in the sample; b) incubating the fixed bacteria with at least one oligonucleotide as described herein and above under (i)-(iv) and elsewhere herein, in order to achieve hybridization; c) removing non-hybridized oligonucleotides; and d) detecting and visualizing the filamentous bacterial cells with the hybridized oligonucleotides. The oligonucleotide can be operatively linked to a detectable marker, including, for example, a) fluorescent marker, b) chemoluminescent marker, c) radioactive marker, d) enzymatically active groups e) hapten, and f) nucleic acids detectable by hybridization. The sample can be an activated sludge sample. The methods can further include quantifying the filamentous bacterial cells with the hybridized oligonucleotides. Also, detection can be performed by epifluorescence microscopy or flow cytometry.

The filamentous bacteria can belong to bacteria as described herein, including, 021N Kanagawa group I, 021N Kanagawa group II, 021N Kanagawa group III, 021N like from BIO33 EU21, Alisphaera europaea EU24 Nostocoida limicola-like, Alisphaera (europaea, PPx3, MC2), Alisphaera MC2 MACOBS-clone 2 (BIO36), Bactothrix amylovora (EU3, EU4, EU8, EU9, EU11), Chloroflexus aurantiacus, Curtunema variabilis (Type 0041), Cytophaga, EPT5 australian 021N isolate (EU21), EPT5 australian 021N isolate, EU23 from SAN3, Flexibacter, Herpetosiphon, Herpetosiphon aurantiacus, Leptothrix discophora, Megathrix sidereus EU26 Nostocoida/021N-like, Megathrix tenacis (EU12, EU5, EU6, EU15, EU13, EU14), (EU1, EU2, EU10), Nostocoida limicola (EU24), Nostocoida limicola-like Rhodobacter sphaeroides next relative, Thiothrix 021N-group und EU1, EU2, EU10), Thiothrix ramosa, type 0411 (CF), type 0803, and Nostocoida limicola-like filamentous bacterium.

Still further embodiments relate to methods for the detection of filamentous bacteria in a sample, using an oligonucleotide as described herein.

Other embodiments relate to kits for performing the method as described herein. The kits can contain at least one oligonucleotide from (i)-(iv) above, or as described herein. The kits can contain at least one oligonucleotide in a hybridization solution. Also, the kits further can contain washing solution. Furthermore, the kits can also include one or more fixation solutions. The kits further can include a cell breaking solution or enzyme solution.

### DETAILED DESCRIPTION OF THE PREFERRED EMBODIMENT

A unique approach to combine the specificity of the molecular biological methods such as PCR with the possibility to visualize bacteria as represented by the antibody method without accepting the disadvantages involved in the respective method, is the method of fluorescent in situ hybridization (FISH; Amann, R. I., W. Ludwig, and K.-H. Schleifer, Phylogenetic identification and in situ detection of individual microbial cells without cultivation, Microbiol. Rev. (1995) 59:143-169). Hereby, bacterial species, genera or groups can be visualized and identified highly specifically, and, if needed, also an exact quantification can be carried out.

The FISH technique is based on the fact that there are certain molecules present in bacterial cells, which due to their vital function have been mutated only to a small degree in the course of evolution. These are the 16S and the 23S ribosomal ribonucleic acids (rRNA). Both are constituents of the ribosomes, the sites of protein biosynthesis, and can serve as specific markers, due to their ubiquitous distribution, their size and their structural and functional constancy (Woese, C. R., Bacterial evolution, Microbiol. Rev. (1987) 51:221-271).

Based on a comparative sequence analysis, phylogenetic relations can be derived solely from these data. For this, these sequence data have to be aligned. In an alignment, which is based on knowledge of the secondary and tertiary structures of these macromolecules, the homologous positions of the ribosomal nucleic acids are correlated.

Based on these data, phylogenetic calculations can be performed. Using state-of-the-art computer technology allows fast and efficient calculations, even if they are large-scale, as well as the establishment of large databases containing the aligned sequences of the 16S rRNA and 23S rRNA. Through fast access to this data material, newly obtained sequences can be analyzed phylogenetically in a short period of time. These rRNA databases can be used to construct specific gene probes. Hereby, all available rRNA sequences are compared and probes are designed for certain sequence parts, which specifically detect a bacterial species, genus or group.

In FISH (fluorescence in situ hybridization), these gene probes, which are complementary to a certain region on the ribosomal target sequence, are introduced into the cell. Usually, the gene probes are small, 16-20 bases long, single-stranded deoxyribonucleic acid fragments, and are directed to a target region, which is typical for a bacterial species or a bacterial group. If the fluorescence-labeled gene probe finds its target sequence in a bacterial cell, so it binds thereto, and the cells can be detected due to their fluorescence in the fluorescence microscope.

As already indicated above, culture-dependent methods give only a very biased insight into the composition and dynamics of the microbial biocoenosis. Using the FISH technique it could be demonstrated that, for example, in detecting activated sludge flora, cultivation results in a cultivation shift (Wagner, M., R. Amann, H. Lemmer, and K. H. Schleifer, Probing activated sludge with oligonucleotides specific for proteobacteria: inadequacy of culture-dependent methods for describing microbial community structure, AppL. Environ. Microbiol. (1993) 59:1520-1525).

By this medium-dependent biasing of the real bacterial community structures, the importance of bacteria that play a subordinate role in activated sludge but have adapted well to the used cultivation conditions, is dramatically overestimated. Thus it could be demonstrated that due to such a cultivation artifact, the bacterial genus Acinetobacter has been completely incorrectly evaluated regarding its role as a biological phosphate removal in wastewater treatment. As a result of such erroneous evaluations, cost-intensive, flawed or imprecise plants are designed. The efficiency and reproducibility of such simulation calculations is small.

The advantages of the FISH technique compared to the identification of bacteria using cultivation are manifold. Firstly, many more cells can be detected using gene probes. Whereas maximally only 15% of the bacterial population of a sample can be visualized by cultivation, FISH allows detection of up to 100% of the total bacterial population in many samples. Secondly, the active part of community can be determined by the ratio between the probe, which is directed to all bacteria and an unspecific cell staining. Thirdly, the bacteria are made visible directly at the spot where they function (in situ). Thus, possible interactions between various bacterial populations can be recognized and analyzed. Fourthly, the detection of bacteria using the FISH technique is much faster than using cultivation. Whereas identification of bacteria using cultivation frequently requires several days, the time from taking a sample to identifying the bacteria, even on the species level, takes only a few hours using the FISH technique. Fifthly, gene probes can be selected almost without restriction with regard to their specificity. Individual species can be detected with one probe as well as an entire genera or bacterial groups. Sixthly, bacterial species or entire bacterial populations can be exactly quantified directly in the sample. Cultivation and the associated insufficient quantification are not necessary.

FISH technology is also superior to conventional staining methods (Gram's or Neisser's stains). Especially, the attempt to characterize filamentous bacteria taken from industrial sewage treatment plants by these conventional techniques must be considered unsuccessful. Numerous bacteria have initially been assigned to the species Nostocoida limicola type II on account of their morphology and staining behavior. In this case it concerns Gram-positive bacteria having a high G+C content in their DNA (Blackall et al., ‘Candidatus Nostocoida limicola’, a filamentous bacterium from activated sludge, Int. J Syst. Evolut. Microbiol. (2000) 50:703-709).

In turn, analysis of these filamentous bacteria using FISH technique gave that most of these bacteria don't have common characteristics with those filamentous bacteria designated as Nostocoida limicola type II. In most cases said filamentous bacteria from industrial sewage treatment plants which first were wrongly classified as Nostocoida limicola belonged to the alpha-subclass of Proteobacteria.

Thus, the FISH technique is a superior tool for fast and highly specific detection of bacteria, directly in a sample. In contrast to cultivation methods, it is a direct procedure and allows, in contrast to other modern methods, not only the visualization of the bacteria but in addition their exact quantification.

In principle, the FISH analysis is performed on a slide, since the bacteria are visualized, i.e. are made visible, during evaluation by irradiation with high-energy light.

The performance of the method of the present invention for specific and fast detection of filamentous bacteria, for example in activated sludge samples, comprises the following steps:

fixing the bacteria contained in the sample,

incubating the fixed bacteria with nucleic acid probe molecules in order to achieve hybridization,

removing or washing off the non-hybridized nucleic acid probe molecules and

detecting the bacteria hybridized with the nucleic acid probe molecules.

Within the scope of the present invention “fixing” of the bacteria means a treatment with which the cell envelope of the bacteria is made permeable for nucleic acid probes. Ethanol is usually used for fixation. If the cell wall cannot be penetrated by the nucleic acid probes using these measures, one or ordinary skill in the art will know sufficient further measures which lead to the same result. These include, for instance, methanol, mixtures of alcohols, a low percentage paraformaldehyde solution or a diluted formaldehyde solution, or the like. Enzymatic steps may be followed in order to cause complete disintegration of the bacteria. Enzymes, which can be used for this step are for instance lysozyme, proteinase K and mutanolysine. One of ordinary skill in the art will know sufficient further techniques and will easily find out which agent is especially useful for cell disintegration, depending on which bacteria is involved.

Within the scope of the present invention the fixed bacteria are incubated for the “hybridization” using fluorescence-labeled nucleic acid probes. These nucleic acid probes, consisting of an oligonucleotide and a marker linked thereto, are then able to penetrate the cell envelope in order to bind to the target sequence corresponding to the nucleic acid probe within the cell. The binding is to be understood as a formation of hydrogen bonds among complementary nucleic acid regions.

The nucleic acid probe may hereby be complementary to a chromosomal or episomal DNA, but also to an MRNA or rRNA of the microorganism to be detected. It is advantageous to select a nucleic acid probe that is complementary to a region present in copies of more than 1 in the microorganism to be identified. The sequence to be detected is preferably present in 500-100,000 copies per cell, especially preferred in 1,000-50,000 copies. For this reason, the rRNA is used preferably as a target site, since in each active cell the ribosomes as sites of protein biosynthesis are present in many thousand copies.

The nucleic acid probe within the meaning of the invention may be a DNA or RNA probe comprising usually between 12 and 1,000 nucleotides, preferably between 12 and 500, more preferably between 12 and 200, especially preferably between 12 and 50 and between 12 and 40, and most preferably between 17 and 25 nucleotides. The selection of the nucleic acid probes is done according to the criteria of whether a complementary sequence is present in the microorganism to be detected. By selecting a defined sequence, a bacterial species, a bacterial genus or an entire bacterial group may be detected. In a probe consisting of 15 nucleotides, 100% of the sequence should be complementary. In oligonucleotides consisting of more than 15 nucleotides, one or more mismatches are allowed.

Compliance with stringent hybridization conditions ensures that the nucleic acid molecule will indeed hybridize with the target sequence. Stringent conditions within the meaning of the invention are for example 20-80% formamide in the hybridization buffer as will be even illustrated in the following. Moreover, stringent hybridization conditions may of course also be looked up in the literature and standard works of reference (such as the Manual of Sambrook et al. (1989) Cold Spring Harbor Laboratory Press, Cold Spring Harbor, N.Y.).

In the context of the method according to the invention, the inventive nucleic probe molecules comprise the lengths and sequences as set out below (all nucleic acid probe molecules are noted in 5′ to 3′ direction).

The nucleic acid probe molecules of the present invention are useful for the detection of the following threadlike, so-called filamentous bacteria:

021N Kanagawa group I, 021N Kanagawa group II, 021N Kanagawa group III, 021N like from BIO33 EU21, Alisphaera europaea EU24 Nostocoida limicola-like, Alisphaera (europaea, PPx3, MC2), Alisphaera MC2 MACOBS-clone 2 (BIO36), Bactothrix amylovora (EU3, EU4, EU8, EU9, EU11), Chloroflexus aurantiacus, Curtunema variabilis (type 0041), Cytophaga, EPT5 australian 021N isolate (EU21), EPT5 australian 021N isolate, EU23 from SAN3, Flexibacter, Herpetosiphon, Herpetosiphon aurantiacus, Leptothrix discophora, Megathrix sidereus EU26 Nostocoida/021N-like, Megathrix tenacis (EU12, EU5, EU6, EU15, EU13, EU14), Nostocoida limicola (EU24), Nostocoida limicola-like Rhodobacter sphaeroides next relative, Thiothrix (021N-group and EU1, EU2, EU10), Thiothrix ramosa, type 0411 (CF), type 0803, Nostocoida limicola-like filamentous bacterium.

| | |
|---|---|
| Sequence 5′→3′ | |
| ACC AGC CCC TGA TAC CCT | (SEQ ID NO:1) |
| AAG GTT CGC CCA CCG ACT | (SEQ ID NO:2) |
| CCG ACA CTA CCC ACT CGT | (SEQ ID NO:3) |
| TCT CAC CCT CAA GAT CGC | (SEQ ID NO:4) |
| GCT GCA CCA CCA ATC TCT | (SEQ ID NO:5) |
| AAG CCC CTC CCG ATT CCA | (SEQ ID NO:6) |
| ACC TAC CTC CAG AGC ATT | (SEQ ID NO:7) |
| CCC TCC CGA TTC CAT AAA | (SEQ ID NO:8) |
| CAA ATA GGG GCA GGT TGC | (SEQ ID NO:9) |
| TGG CCC ACC GGC TTC GGG | (SEQ ID NO:10) |
| ACC CTC CTC TCC CGG TCT | (SEQ ID NO:11) |
| ACC AGC CTC CAC TTC TCT | (SEQ ID NO:12) |
| TAC CTT CCG CTT TAG GTC | (SEQ ID NO:13) |
| TCG GSC GCT CCG TGA GCG | (SEQ ID NO:14) |
| CCG TGA GCG CAA GGC CTT | (SEQ ID NO:15) |
| ACG TTC CTC TGC GAG CCT | (SEQ ID NO:16) |
| GGC ACG GAA CGA CGC GAA | (SEQ ID NO:17) |
| CTC TCC TCA CCT CTA GTC | (SEQ ID NO:18) |
| CCT CTC CTC GCC TCA A | (SEQ ID NO:19) |
| TCA CGG ACT TCA GGC GTT | (SEQ ID NO:20) |
| CTC AGT AGA TTC CCA CGT | (SEQ ID NO:21) |
| GCG GTT AGC CTA GCT ACT | (SEQ ID NO:22) |
| TGG TAA CCG GCC TCC TTG | (SEQ ID NO:23) |
| TAA AGC GAG ACT GAC GGC | (SEQ ID NO:24) |
| TGC CGC ACT CCA GCT ATA | (SEQ ID NO:25) |
| GCC GCA CTC CAG CTA TAC | (SEQ ID NO:26) |
| CTC TCC CGG ACT CGA GCC | (SEQ ID NO:27) |
| TCT CGA CCT CAA GAA CAG | (SEQ ID NO:28) |
| ACT TCC CTC TCC CAA ATT | (SEQ ID NO:29) |
| GCG ACT TGC GCC TTT CCC | (SEQ ID NO:30) |
| GCT GCA CCA CCG ACC CCT | (SEQ ID NO:31) |
| TGC CGC ACT CCA GCG ATG | (SEQ ID NO:32) |
| ACT TCC CTC TCC CAC ATT | (SEQ ID NO:33) |
| CCT TCC GAT CTC TAT GCA | (SEQ ID NO:34) |
| CCT TCC GAT CTC TAC GCA | (SEQ ID NO:35) |
| TGT GTT CGA GTT CCT TGC | (SEQ ID NO:36) |
| GCA CCA CCG ACC CCT TAG | (SEQ ID NO:37) |
| CTC AGG GAT TCC TGC CAT | (SEQ ID NO:38) |
| TCG CCT CTC TCA TCC TC | (SEQ ID NO:39) |
| TCC GGT CTC CAG CCA CA | (SEQ ID NO:40) |
| AAG TCC CCC GAC ATC CAG | (SEQ ID NO:41) |
| ACC CGA CCG TGG ACG GCT | (SEQ ID NO:42) |

In the sequences “S” stands for “G+C”.

The subject of the invention also comprises modifications of the aforementioned oligonucleotide sequences, which despite the modifications in the sequence and/or length show specific hybridization with target nucleic acid sequences of the respective bacterium and thus are useful for application in a method according to the invention. These especially include:

a) nucleic acid molecules, (i) being identical to one of the above oligonucleotide sequences (SEQ ID No. 1 to SEQ ID No. 42) in at least 60%, 65%, preferably in at least 70%, 75%, more preferably in at least 80%, 84%, 87% and particularly preferably in at least 90%, 94%, 96% of the bases (wherein the sequence region of the nucleic acid molecule corresponding to the sequence region of one of the oligonucleotides given above (SEQ ID Nos. 1 to 42) is to be considered, and not the entire sequence of a nucleic acid molecule, which possibly may be longer in sequence compared to the oligonucleotides given above (SEQ ID No. 1 to SEQ ID No. 42) by one or numerous bases) or (ii) differing from the above oligonucleotide sequences (SEQ ID No. 1 to SEQ ID No. 42) by one or several deletions and/or additions, and allowing for specific hybridization with nucleic acid sequences of filamentous bacteria. “Specific hybridization” hereby means that under the hybridization conditions described here, or those known to the person skilled in the art in the context of in situ hybridization techniques, only the ribosomal RNA of target organisms binds to the oligonucleotide and not the rRNA of non-target organisms.

b) nucleic acid molecules, being complementary to the nucleic acid molecules named under a) or to one of the probes SEQ ID No. 1 to SEQ ID No. 42, or specifically hybridizing with them under stringent conditions,

c) nucleic acid molecules comprising an oligonucleotide sequence from SEQ ID No. 1 to SEQ ID No. 42 or comprising the sequence of a nucleic acid molecule according to a) or b) and which, in addition to the sequences mentioned or their modifications according to a) or b), have at least a further nucleotide, and which allow for specific hybridization with nucleic acid sequences of target organisms.

Usual algorithms can determine the degree of sequence identity of a nucleic acid molecule with the probes SEQ ID No. 1 to SEQ ID No. 42. In this respect, for example, the program for the determination of sequence identity, which is accessible under hypertext transfer protocol on the worldwide web at “ncbi.nlm.nih.gov/BLAST” (http://www.ncbi.nlm.nih.gov/BLAST) (on this website there is for example the link “Standard nucleotide-nucleotide BLAST [blastn]”) is suitable here.

In the present invention “hybridization” can have the same meaning as “complementary”. The present invention also comprises those oligonucleotides, which hybridize to the (theoretical) antisense strand of one of the inventive oligonucleotides including also the modifications of SEQ ID Nos. 1 to 42 according to the invention.

It is specifically an advantage of the method according to the invention that now for the first time filamentous bacteria, particularly those occurring in industrial sewage treatment plants, may be detected specifically using this method. Up to now conventional detection methods are often restricted to such filamentous bacteria that are present in municipal waste treatment plants. Microbial populations of municipal sewage treatment plants and industrial sewage treatment plants are however completely different. Conventional methods, which merely allow for (partial) detection of bacteria present in municipal sewage treatment plants, are not meaningfully applicable to industrial plants, since the corresponding bacteria are simply not present there. Using the method or the probes according to the invention it is possible for the first time to apply the advantages of FISH to the industrial wastewater treatment field as well.

Thus, a particular advantage of the invention is that, if desired, all bacteria as mentioned here which cause sludge bulking and skimmings can be simultaneously detected in industrial sewage treatment plants.

Further advantages are that using one or more oligonucleotides of particular probe sub-groups according to the invention, particular sub-groups of target organisms can be selectively detected.

Here, the group of filamentous bacteria, which most commonly cause bulking sludge and skimmings in industrial plants, has to be mentioned first. Nostocoida limicola-like Rhodobacter sphaeroides next relative, Nostocoida limicola (EU24), Alisphaera (europaea, PPx3, MC2), Alisphaera europaea EU24 Nostocoida limicola-like are counted among these bacteria.

This group is identified preferably by using at least one of the oligonucleotides selected from the group consisting of SEQ ID Nos. 27, 40, 41 and 42, and modifications of these oligonucleotides according to the invention.

Another sub-group of bacteria which has to be mentioned is the group to which the bacteria Nostocoida limicola-like filamentous bacterium belong. These bacteria are detected preferably using at least one oligonucleotide according to SEQ ID Nos. 1 to 3. This group is the second most common causer of bulking sludge and skimmings in industrial plants.

The bacteria Bactothrix amylovora (EU3, EU4, EU8, EU9, EU11) also have to be mentioned, which, especially in sewage treatment plants of paper mills, are the main causers of sludge bulking and skimmings. This group of bacteria is detected specifically advantageously by using one or several oligonucleotide probes according to SEQ ID Nos. 6 to 9.

The other causers of sludge bulking and skimmings, such as e.g. Chloroflexus aurantiacus, Curtunema variabilis (type 0041), Cytophaga, EPT5 australian 021N isolate (EU21), EPT5 australian 021N isolate, EU23 from SAN3, Flexibacter, Herpetosiphon, Herpetosiphon aurantiacus, Leptothrix discophora, Megathrix sidereus EU26 Nostocoida/021N-like, Megathrix tenacis (EU12, EU5, EU6, EU15, EU13, EU14), (EU1, EU2, EU10), Thiothrix 021N group and EU1, EU2, EU10), Thiothrix ramosa, type 0411 (CF), type 0803, are detected, preferably by using other oligonucleotide probes provided in the context of the present invention.

In the context of the inventive detection method, the nucleic acid probe molecules according to the invention can be used with various hybridization solutions. For this purpose various organic solvents at concentrations of from 0 to 80% can be used. Compliance with stringent hybridization conditions ensures that the nucleic acid probe molecule will indeed hybridize with the target sequence. Moderate conditions within the meaning of the invention are, e.g. 0% formamide in a hybridization buffer as described below. Stringent conditions within the meaning of the invention are for example 20-80% formamide in the hybridization buffer.

Within the scope of the method according to the invention, a typical hybridization solution contains 0-80% formamide, preferably 20-80% and 20-60% formamide and especially preferably 35% formamide and has a salt concentration of from 0.1 mol/l to 1.5 mol/l, preferably of from 0.5 mol/l to 1.0 mol/l, more preferably of from 0.7 mol/l to 0.9 mol/l and most preferably of 0.9 mol/l, with the salt being preferably sodium chloride. Further, the hybridization solution usually comprises a detergent, such as for instance sodium lauryl sulfate (SDS) at a concentration of from 0.001% to 0.2%, preferably at a concentration of from 0.005-0.05%, more preferably of from 0.01-0.03%, and most preferably 0.01%. The hybridization solution may be buffered with various compounds, such as tris-HCl, sodium citrate, PIPES or HEPES buffer, which are used usually at concentrations of from 0.01 to 0.1 mol/l, preferably of from 0.01 to 0.08 mol/l, preferably in a pH range of from 6.0 to 9.0, especially preferably of from 7.0 to 8.0. The preferred embodiment of the hybridization solution of the present invention contains 0.02 mol/l tris-HCl, pH 8.0.

It shall be understood that the person skilled in the art can select the given concentrations of the components of the hybridization buffer in such a way that the required stringency of the hybridization reaction is achieved. Particularly preferred embodiments reflect stringent to particularly stringent hybridization conditions. Using these stringent conditions, the person skilled in the art can determine whether a given nucleic acid molecule permits the specific detection of nucleic acid sequences of target organisms and can therefore be used reliably in the context of the invention.

The concentration of the nucleic acid probe in the hybridization buffer depends on the type of labeling and the number of the target structures expected. To allow for rapid and efficient hybridization, the number of nucleic acid probe molecules should exceed the number of target structures by several orders of magnitude. On the other hand, it needs to be considered when working with fluorescence in situ hybridization (FISH) that an excessively high level of fluorescently labeled nucleic acid probe molecules leads to an increase in background fluorescence. The concentration of the nucleic acid probe molecules should therefore be in the range of 0.5 ng/μl and 500 ng/μl, preferably between 1.0 ng/μl and 100 ng/μl and particularly preferably in the range of 1.0 ng/μl and 50 ng/μl.

In the context of the method of the present invention, the preferred concentration is 1-10 ng of each nucleic acid probe molecule used per μl hybridization solution. The used volume of the hybridization solution should be between 8 μl and 100 μl; in a particularly preferred embodiment of the method of the present invention it is 40 μl.

The duration of the hybridization is normally between 10 minutes and 12 hours; the hybridization preferably lasts for about 1.5 hours. The hybridization temperature is preferably between 44° C. and 48° C., particularly preferably 46° C., whereby the parameter of the hybridization temperature as well as the concentration of salts and detergents in the hybridization solution can be optimized based on the nucleic acid probes, in particular their lengths and the degree to which they are complementary to the target sequence in the cell to be detected. The person skilled in the art is familiar with the pertinent calculations. The above-described conditions represent stringent hybridization conditions.

After completion of hybridization, the non-hybridized and excess nucleic acid probe molecules should be removed or washed off, which is usually accomplished by a conventional washing solution. If desired, this washing solution may contain 0.001-0.1% of a detergent such as SDS, a concentration of 0.01% being preferred, as well as tris-HCl in a concentration of 0.001-0.1 mol/l, preferably 0.01-0.05 mol/l, most preferably 0.02 mol/l, the pH value of tris-HCl being in the range of from 6.0 to 9.0, preferably between 7.0 and 8.0, and most preferably at 8.0. A detergent can be included, but it is not mandatory. The washing solution also usually contains NaCl at a concentration depending on the required stringency, of from 0.003 mol/l to 0.9 mol/l, preferably of from 0.01 mol/l to 0.9 mol/l. An NaCl concentration of 0.07 mol/l is particularly preferred. In addition, the washing solution may contain EDTA, the concentration preferably being 0.005 mol/l. The washing solution can further contain suitable quantities of commonly used preservatives which are known to the person skilled in the art.

In general, buffer solutions are used during the washing step, which can in principle be very similar to the hybridization buffer (buffered sodium chloride solution), the only difference being that the washing step is performed in a buffer with a lower salt concentration or at higher temperature.

The following equation can be used for the theoretical estimation of the hybridization conditions:

Td=81.5+16.6 1g[Na+]+0.4×(% GC)−820/n−0.5×(% FA)

Td=dissociation temperature in ° C.

[Na+]=molarity of sodium ions

% GC=proportion of guanine and cytosine nucleotides relative to the number of total bases

n=hybrid length

% FA=formamide content

Using this equation, for example the proportion of formamide in the washing buffer (which should be kept as low as possible because of formamide's toxicity) can be replaced with a correspondingly lower content of sodium chloride. However, the person skilled in the art is aware, on the basis of the extensive literature on in situ hybridization methods, that these components can be varied as well as how they can be varied. The above remarks with respect to hybridization buffers also apply to the stringency of the hybridization conditions.

The “washing off” of the unbound nucleic acid probe molecules is normally accomplished at temperatures in the range of from 44° C. to 52° C., preferably of from 44° C. to 50° C. and particularly preferably at 46° C. for a duration of 10-40 minutes, preferably for 15 minutes.

In an alternative embodiment of the method of the present invention, the nucleic acid probe molecules according to the invention are used in the so-called Fast FISH method for specifically detecting the given target organisms. The Fast FISH method is known to the person skilled in the art and is, for example, described in German patent application DE 199 36 875.9 and in international application WO 99/18234. Explicit reference is made here to the disclosure for performing the detection method described in these documents.

The specifically hybridized nucleic acid probe molecules can then be detected in the corresponding cells, provided that the nucleic acid probe molecule is detectable, for instance in that the nucleic acid probe molecule is covalently linked to a marker. Detectable markers which are used and which are all well known to the person skilled in the art include fluorescent groups such as CY2 (available from Amersham Life Sciences, Inc., Arlington Heights, USA), CY3 (also available from Amersham Life Sciences), CY5 (also available from Amersham Life Sciences), FITC (Molecular Probes Inc., Eugene, USA), FLUOS (available from Roche Diagnostics GmbH, Mannheim, Germany), TRITC (available from Molecular Probes Inc., Eugene, USA). 6FAM or FLUOS-PRIME. Chemical markers, radioactive markers or enzymatic markers such as horseradish peroxidase, acid phosphatase, alkaline phosphatase and peroxidase can be used as well. A series of chromogens is known for each of these enzymes, which can be reacted instead of the natural substrate, forming colored or fluorescent products. Examples of such chromogens are given in the following Table:

TABLE 1

| Enzyme | | Chromogen |
|---|---|---|
| 1. | Alkaline phosphatase | 4-methylumbelliferylphosphate (*), |
| | and acid phosphatase | bis(4-methylumbelliferylphosphate), (*) 3-O- |
| | | methylfluorescein, flavone-3- |
| | | diphosphate triammonium salt (*), |
| | | p-nitrophenylphosphate disodium salt |
| 2. | Peroxidase | tyramine hydrochloride (*), 3-(p-hydroxy- |
| | | phenyl)-propionic acid (*), p-hydroxy- |
| | | phenethylalcohol (*), 2,2′-azino-di-3- |
| | | ethylbenzthiazolinesulfonic acid (ABTS), |
| | | ortho-phenylendiamine dihydrochloride, |
| | | o-dianisidine, 5-aminosalicylic acid, |
| | | p-ucresol (*), 3,3′-dimethyloxybenzidine, |
| | | 3-methyl-2-benzothiazoline hydrazone, |
| | | tetramethylbenzidine |
| 3. | Horseradish | H₂O₂ + diammonium benzidine |
| | peroxidase | H₂O₂ + tetramethylbenzidine |
| 4. | β-D-galactosidase | o-Nitrophenyl-β-D-galactopyranoside, |
| | | 4-methylumbelliferyl-β-D-galactoside |
| 5. | Glucose oxidase | ABTS, glucose and thiazolyl blue |

*Fluorescence

Finally, it is possible to form nucleic acid probe molecules in such a way that there is a further nucleic acid sequence at their 5′ or 3′ end, which is also suitable for hybridization. This nucleic acid sequence in turn includes approximately 15 to 1,000, preferably 15-50 nucleotides. This second nucleic acid region can then be recognized by a nucleic acid probe molecule, which is detectable by any of the agents given above.

Another possibility is the coupling of the detectable nucleic acid probe molecule to a hapten. The nucleic acid probe molecule can then be brought into contact with antibodies, which recognize the hapten. An example of such a hapten is digoxigenin. Further examples besides those mentioned above are well known to the person skilled in the art.

The final analysis depends on the type of labeling of the used probe and can be conducted using an optical microscope, an epifluoresence microscope, chemoluminometer, fluorometer or the like.

The method of the present invention enables for the first time the definite and specific detection of filamentous bacteria as mentioned above and thus the first-time differentiation between bacterial filaments, whereas classical methods (i.e. for example morphological characterization or Gram's or Neisser's stain) could not draw a clear dividing line between them.

An important advantage of the method described in this application for the specific fast detection of filamentous bacteria, for example in activated sludge samples, is its speed compared to conventional detection methods as described above. Results using the method of the present invention are available within 3 hours.

Another advantage is the specificity of this method. With the used nucleic acid probe molecules, entire genera or groups can be detected and visualized specifically as well as single species from these genera can be detected and visualized highly specifically. Through visualization of the bacteria, a visual control may be carried out at the same time. False positive results are thus excluded.

Another advantage of the method according to the invention is the opportunity of simultaneous and specific detection of most different filamentous bacteria. The use of variously labeled nucleic acid probe molecules renders it possible easily and reliably.

Another advantage of the inventive method is the opportunity, arising from the visualization of the bacteria, of easy and accurate quantification of the bacteria contained in the sample.

A further advantage of the inventive method is its ease of handling, so that large amounts of samples may easily be tested for the presence of the mentioned bacteria.

The method of the present invention can be applied manifold. Besides analysis of samples from activated sludge the method may also be used for analysis of a variety of other environmental samples which are taken from air, water or soil.

According to the invention, in a further aspect of the invention, a kit for applying the method according to the invention is provided. The hybridization arrangement contained in these kits is, for instance, described in German patent application 100 61 655.0. It is expressly referred to the disclosure contained in this document concerning the in situ hybridization arrangement.

Besides the described hybridization arrangement (called VIT reactor), the most important component of the kits is their respective hybridization solution containing the specific nucleic acid probe molecules for the microorganisms to be detected, as described above (so-called VIT solution). The kits also always contain the corresponding hybridization buffer (Solution C) and a concentrate of the corresponding washing solution (Solution D). The kit may also contain fixation solutions (Solution A and Solution B) if needed, and additionally a cell breaking solution (Breaker_{—}2) as well as, if needed, an embedding solution (finisher). Finishers are commercially available and their activity also includes the prevention of rapid bleaching of fluorescent probes under the fluorescent microscope. Optionally, solutions for parallel performance of a positive control and a negative control may also be included.

The following example is intended to describe the invention, however without limiting it:

### EXAMPLE

Specific fast detection of filamentous bacteria in samples, e.g., taken from activated sludge

An appropriate aliquot of the sample material to be analyzed is applied onto a slide and dried (46° C., 30 min or until completely dry).

The dried cells are then dehydrated stepwise.

Therefore, first of all a fixation solution (Solution A, 50% ethanol) is applied, whereby 40 μl are preferred. The slide is again dried (46° C., 30 min or until totally dry).

After that the dried cell are completely dehydrated by adding a further fixation solution (Solution B (absolute ethanol), preferably 40 μl). The slide is dried again (room temperature, 3 min or until totally dry). For complete disintegration of the cells, a suitable volume of a suitable enzyme solution (Breaker, 40 μl are preferred) can be applied onto the slide and the slide is then incubated (10 to 30 min, 4-25° C.).

The enzyme solution is washed off by immersing the slide in a tube, preferably the VIT reactor, filled with distilled water and the slide is then dried in a lateral position (46° C., 30 min or until completely dry).

Thereinafter the hybridization solution (VIT solution), comprising the specific nucleic acid probe molecules described above for each of the microorganisms to be detected, is applied onto the fixed dehydrated cells. The preferred volume is 40 μl. The slide is then incubated (46° C., 90 min) within a chamber, preferably the VIT reactor, which is moistened with hybridization buffer (Solution C, which corresponds to the hybridization solution without oligonucleotide).

The slide is then removed from the chamber, preferably the VIT reactor, and the chamber, again preferably the VIT reactor, is filled with a washing solution (Solution D, diluted 1:10 in distilled water) and the slide is incubated therein (46° C., 15 min).

After that the VIT reactor is filled with distilled water, the slide is immersed therein for a short period of time and the slide is then dried in a lateral position (46° C., 30 min or until completely dry).

To the end the slide is embedded in a suitable medium (finisher). Finally, the sample is analyzed using a fluorescence microscope.

What is claimed is:

1. An isolated oligonucleotide selected from the group consisting of:
(i) an oligonucleotide having one of the following nucleotide sequences
ACC AGC CCC TGA TAC CCT,
AAG GTT CGC CCA CCG ACT,
CCG ACA CTA CCC ACT CGT,
TCT CAC CCT CAA GAT CGC,
GCT GCA CCA CCA ATC TCT,
AAG CCC CTC CCG ATT CCA,
ACC TAC CTC CAG AGC ATT,
CCC TCC CGA TTC CAT AAA,
CAA ATA GGG GCA GGT TGC,
ACC AGC CTC CAC TTC TCT,
TAC CTT CCG CTT TAG GTC,
TCG GSC GCT CCG TGA GCG,
CCG TGA GCG CAA GGC CTT,
ACG TTC CTC TGC GAG CCT,
GGC ACG GAA CGA CGC GAA,
CTC TCC TCA CCT CTA GTC,
CCT CTC CTC GCC TCA A,
TCA CGG ACT TCA GGC GTT,
CTC AGT AGA TTC CCA CGT,
GCG GTT AGC CTA GCT ACT,
TGG TAA CCG GCC TCC TTG,
TAA AGC GAG ACT GAC GGC,
TGC CGC ACT CCA GCT ATA,
GCC GCA CTC CAG CTA TAC,
CTC TCC CGG ACT CGA GCC,
TCT CGA CCT CAA GAA CAG,
ACT TCC CTC TCC CAA ATT,
GCG ACT TGC GCC TTT CCC,
GCT GCA CCA CCG ACC CCT,
TGC CGC ACT CCA GCG ATG,
ACT TCC CTC TCC CAC ATT,
TCG CCT CTC TCA TCC TC,
TCC GGT CTC CAG CCA CA,
AAG TCC CCC GAC ATC CAG,
ACC CGA CCG TGG ACG GCT,
(ii) an oligonucleotide being at least 80% identical to one of the oligonucleotides from (i), and rendering possible specific hybridization with nucleic acid sequences of filamentous bacterial cells,
(iii) an oligonucleotide differing from one of the oligonucleotides from (i) by a deletion and/or addition and rendering possible specific hybridization with nucleic acid sequences of filamentous bacterial cells, and
(iv) an oligonucleotide hybridizing under stringent conditions with one of the oligonucleotides from (i), (ii) or (iii).

2. A method for detecting filamentous bacteria in a sample, comprising the steps:
a) fixing the filamentous bacteria contained in the sample;
b) incubating the fixed bacteria with at least one oligonucleotide, selected from the group consisting of
(i) an oligonucleotide having one of the following nucleotide sequences
ACC AGC CCC TGA TAC CCT,
AAG GTT CGC CCA CCG ACT,
CCG ACA CTA CCC ACT CGT,
TCT CAC CCT CAA GAT CGC,
GCT GCA CCA CCA ATC TCT,
AAG CCC CTC CCG ATT CCA,
ACC TAC CTC CAG AGC ATT,
CCC TCC CGA TTC CAT AAA,
CAA ATA GGG GCA GGT TGC,
TGG CCC ACC GGC TTC GGG,
ACC CTC CTC TCC CGG TCT,
ACC AGC CTC CAC TTC TCT,
TAC CTT CCG CTT TAG GTC,
TCG GSC GCT CCG TGA GCG,
CCG TGA GCG CAA GGC CTT,
ACG TTC CTC TGC GAG CCT,
GGC ACG GAA CGA CGC GAA,
CTC TCC TCA CCT CTA GTC,
CCT CTC CTC GCC TCA A,
TCA CGG ACT TCA GGC GTT,
CTC AGT AGA TTC CCA CGT,
GCG GTT AGC CTA GCT ACT,
TGG TAA CCG GCC TCC TTG,
TAA AGC GAG ACT GAC GGC,
TGC CGC ACT CCA GCT ATA,
GCC GCA CTC CAG CTA TAC,
CTC TCC CGG ACT CGA GCC,
TCT CGA CCT CAA GAA CAG,
ACT TCC CTC TCC CAA ATT,
GCG ACT TGC GCC TTT CCC,
GCT GCA CCA CCG ACC CCT,
TGC CGC ACT CCA GCG ATG,
ACT TCC CTC TCC CAC ATT,
CCT TCC GAT CTC TAT GCA,
CCT TCC GAT CTC TAC GCA,
TGT GTT CGA GTT CCT TGC,
GCA CCA CCG ACC CCT TAG,
CTC AGG GAT TCC TGC CAT,
TCG CCT CTC TCA TCC TC,
TCC GGT CTC CAG CCA CA,
AAG TCC CCC GAC ATC CAG,
ACC CGA CCG TGG ACG GCT,
(ii) an oligonucleotide being at least 80% identical to one of the oligonucleotides from (i), and rendering possible specific hybridization with nucleic acid sequences of filamentous bacterial cells,
(iii) an oligonucleotide differing from one of the oligonucleotides from (i) by a deletion and/or addition, and rendering possible specific hybridization with nucleic acid sequences of filamentous bacterial cells, and
(iv) an oligonucleotide hybridizing under stringent conditions with one of the oligonucleotides from (i), (ii) or (iii), in order to achieve hybridization,
c) removing non-hybridized oligonucleotides; and
d) detecting and visualizing the filamentous bacterial cells with the hybridized oligonucleotides.

3. The method according to claim 2, wherein detection is performed by epifluorescence microscopy or flow cytometry.

4. The method according to claim 2, wherein the oligonucleotide is operatively linked to a detectable marker, selected from the group consisting of
a) fluorescent marker;
b) chemoluminescent marker;
c) radioactive marker;
d) enzymatically active groups;
e) hapten; and
f) nucleic acids detectable by hybridization.

5. The method according to claim 4, wherein detection is performed by epifluorescence microscopy or flow cytometry.

6. The method according to claim 2, wherein the sample is an activated sludge sample.

7. The method according to claim 6, wherein detection is performed by epifluorescence microscopy or flow cytometry.

8. The method according to claim 2, wherein the filamentous bacteria belong to bacteria of the following designations:
021 N Kanagawa group I, 021N Kanagawa group II, 021N Kanagawa group III, 021N like from BIO33 EU21, Alisphaera europaea EU24 Nostocoida limicola-like, Alisphaera (europaea, PPx3, MC2), Alisphaera MC2 MACOBS-clone 2 (BIO36), Bactothrix amylovora (EU3, EU4, EU8, EU9, EU11), Chloroflexus aurantiacus, Curtunema variabilis (Type 0041), Cytophaga, EPT5 australian 021N isolate (EU21), EPT5 australian 021N isolate, EU23 from SAN3, Flexibacter, Herpetosiphon, Herpetosiphon aurantiacus, Leptothrix discophora, Megathrix sidereus EU26 Nostocoida/021N-like, Megathrix tenacis (EU12, EU5, EU6, EU15, EU13, EU14), (EU1, EU2, EU10), Nostocoida limicola (EU24), Nostocoida limicola-like Rhodobacter sphaeroides next relative, Thiothrix 021N-group und EU1, EU2, EU10), Thiothrix ramosa, type 0411 (CF), type 0803, and Nostocoida limicola-like filamentous bacterium.

9. The method according to claim 2, further comprising quantifying the filamentous bacterial cells with the hybridized oligonucleotides.

10. A method for the detection of filamentous bacteria in a sample, using an oligonucleotide according to claim 1.

11. A kit for performing the method according to claim 2, containing at least one oligonucleotide, selected from the group consisting of
(i) an oligonucleotide having one of the following nucleotide sequences
ACC AGC CCC TGA TAC CCT,
AAG GTT CGC CCA CCG ACT,
CCG ACA CTA CCC ACT CGT,
TCT CAC CCT CAA GAT CGC,
GCT GCA CCA CCA ATC TCT,
AAG CCC CTC CCG ATT CCA,
ACC TAC CTC CAG AGC ATT,
CCC TCC CGA TTC CAT AAA,
CAA ATA GGG GCA GGT TGC,
TGG CCC ACC GGC TTC GGG,
ACC CTC CTC TCC CGG TCT,
ACC AGC CTC CAC TTC TCT,
TAC CTT CCG CTT TAG GTC,
TCG GSC GCT CCG TGA GCG,
CCG TGA GCG CAA GGC CTT,
ACG TTC CTC TGC GAG CCT,
GGC ACG GAA CGA CGC GAA,
CTC TCC TCA CCT CTA GTC,
CCT CTC CTC GCC TCA A,
TCA CGG ACT TCA GGC GTT,
CTC AGT AGA TTC CCA CGT,
GCG GTT AGC CTA GCT ACT,
TGG TAA CCG GCC TCC TTG,
TAA AGC GAG ACT GAC GGC,
TGC CGC ACT CCA GCT ATA,
GCC GCA CTC CAG CTA TAC,
CTC TCC CGG ACT CGA GCC,
TCT CGA CCT CAA GAA CAG,
ACT TCC CTC TCC CAA ATT,
GCG ACT TGC GCC TTT CCC,
GCT GCA CCA CCG ACC CCT,
TGC CGC ACT CCA GCG ATG,
ACT TCC CTC TCC CAC ATT,
CCT TCC GAT CTC TAT GCA,
CCT TCC GAT CTC TAC GCA,
TGT GTT CGA GTT CCT TGC,
GCA CCA CCG ACC CCT TAG,
CTC AGG GAT TCC TGC CAT,
TCG CCT CTC TCA TCC TC,
TCC GGT CTC CAG CCA CA,
AAG TCC CCC GAC ATC CAG,
ACC CGA CCG TGG ACG GCT;
(ii) an oligonucleotide being at least 80% identical to one of the oligonucleotides from (i), and rendering possible specific hybridization with nucleic acid sequences of filamentous bacterial cells;
(iii) an oligonucleotide differing from one of the oligonucleotides from (i) by a deletion and/or addition and rendering possible specific hybridization with nucleic acid sequences of filamentous bacterial cells; and
(iv) an oligonucleotide hybridizing with one of the oligonucleotides from (i), (ii) or (iii) under stringent conditions.

12. The kit according to claim 11, further containing a washing solution.

13. The kit according to claim 12, further comprising one or more fixation solutions.

14. The kit according to claim 12, further comprising a cell breaking solution or enzyme solution.

15. The kit according to claim 11, containing the at least one oligonucleotide in a hybridization solution.

16. The kit according to claim 15, further containing a washing solution.

17. The kit according to claim 16, further comprising one or more fixation solutions.

18. The kit according to claim 16, further comprising a cell breaking solution or enzyme solution.


SEQUENCE LISTING (42 sequences)

SEQ ID NO: 1 - DNA, 18 bp, Artificial Sequence
  Oligonucleotide
accagcccctgataccct

SEQ ID NO: 2 - DNA, 18 bp, Artificial Sequence
  Oligonucleotide
aaggttcgcccaccgact

SEQ ID NO: 3 - DNA, 18 bp, Artificial Sequence
  Oligonucleotide
ccgacactacccactcgt

SEQ ID NO: 4 - DNA, 18 bp, Artificial Sequence
  Oligonucleotide
tctcaccctcaagatcgc

SEQ ID NO: 5 - DNA, 18 bp, Artificial Sequence
  Oligonucleotide
gctgcaccaccaatctct

SEQ ID NO: 6 - DNA, 18 bp, Artificial Sequence
  Oligonucleotide
aagcccctcccgattcca

SEQ ID NO: 7 - DNA, 18 bp, Artificial Sequence
  Oligonucleotide
acctacctccagagcatt

SEQ ID NO: 8 - DNA, 18 bp, Artificial Sequence
  Oligonucleotide
ccctcccgattccataaa

SEQ ID NO: 9 - DNA, 18 bp, Artificial Sequence
  Oligonucleotide
caaataggggcaggttgc

SEQ ID NO: 10 - DNA, 18 bp, Artificial Sequence
  Oligonucleotide
tggcccaccggcttcggg

SEQ ID NO: 11 - DNA, 18 bp, Artificial Sequence
  Oligonucleotide
accctcctctcccggtct

SEQ ID NO: 12 - DNA, 18 bp, Artificial Sequence
  Oligonucleotide
accagcctccacttctct

SEQ ID NO: 13 - DNA, 17 bp, Artificial Sequence
  Oligonucleotide
accttccgctttaggtc

SEQ ID NO: 14 - DNA, 18 bp, Artificial Sequence
  Oligonucleotide
tcggscgctccgtgagcg

SEQ ID NO: 15 - DNA, 18 bp, Artificial Sequence
  Oligonucleotide
ccgtgagcgcaaggcctt

SEQ ID NO: 16 - DNA, 18 bp, Artificial Sequence
  Oligonucleotide
acgttcctctgcgagcct

SEQ ID NO: 17 - DNA, 18 bp, Artificial Sequence
  Oligonucleotide
ggcacggaacgacgcgaa

SEQ ID NO: 18 - DNA, 18 bp, Artificial Sequence
  Oligonucleotide
ctctcctcacctctagtc

SEQ ID NO: 19 - DNA, 16 bp, Artificial Sequence
  Oligonucleotide
cctctcctcgcctcaa

SEQ ID NO: 20 - DNA, 18 bp, Artificial Sequence
  Oligonucleotide
tcacggacttcaggcgtt

SEQ ID NO: 21 - DNA, 18 bp, Artificial Sequence
  Oligonucleotide
ctcagtagattcccacgt

SEQ ID NO: 22 - DNA, 18 bp, Artificial Sequence
  Oligonucleotide
gcggttagcctagctact

SEQ ID NO: 23 - DNA, 18 bp, Artificial Sequence
  Oligonucleotide
tggtaaccggcctccttg

SEQ ID NO: 24 - DNA, 18 bp, Artificial Sequence
  Oligonucleotide
taaagcgagactgacggc

SEQ ID NO: 25 - DNA, 18 bp, Artificial Sequence
  Oligonucleotide
tgccgcactccagctata

SEQ ID NO: 26 - DNA, 18 bp, Artificial Sequence
  Oligonucleotide
gccgcactccagctatac

SEQ ID NO: 27 - DNA, 18 bp, Artificial Sequence
  Oligonucleotide
ctctcccggactcgagcc

SEQ ID NO: 28 - DNA, 18 bp, Artificial Sequence
  Oligonucleotide
tctcgacctcaagaacag

SEQ ID NO: 29 - DNA, 18 bp, Artificial Sequence
  Oligonucleotide
acttccctctcccaaatt

SEQ ID NO: 30 - DNA, 18 bp, Artificial Sequence
  Oligonucleotide
gcgacttgcgcctttccc

SEQ ID NO: 31 - DNA, 18 bp, Artificial Sequence
  Oligonucleotide
gctgcaccaccgacccct

SEQ ID NO: 32 - DNA, 18 bp, Artificial Sequence
  Oligonucleotide
tgccgcactccagcgatg

SEQ ID NO: 33 - DNA, 18 bp, Artificial Sequence
  Oligonucleotide
acttccctctcccacatt

SEQ ID NO: 34 - DNA, 18 bp, Artificial Sequence
  Oligonucleotide
ccttccgatctctatgca

SEQ ID NO: 35 - DNA, 18 bp, Artificial Sequence
  Oligonucleotide
ccttccgatctctacgca

SEQ ID NO: 36 - DNA, 18 bp, Artificial Sequence
  Oligonucleotide
tgtgttcgagttccttgc

SEQ ID NO: 37 - DNA, 18 bp, Artificial Sequence
  Oligonucleotide
gcaccaccgaccccttag

SEQ ID NO: 38 - DNA, 18 bp, Artificial Sequence
  Oligonucleotide
ctcagggattcctgccat

SEQ ID NO: 39 - DNA, 17 bp, Artificial Sequence
  Oligonucleotide
tcgcctctctcatcctc

SEQ ID NO: 40 - DNA, 17 bp, Artificial Sequence
  Oligonucleotide
tccggtctccagccaca

SEQ ID NO: 41 - DNA, 18 bp, Artificial Sequence
  Oligonucleotide
aagtcccccgacatccag

SEQ ID NO: 42 - DNA, 18 bp, Artificial Sequence
  Oligonucleotide
acccgaccgtggacggct